(19) United States
(12) Patent Application Publication
(10) Pub. No.: US 2022/0133182 A1
(43) Pub. Date: May 5, 2022

(54) PORTABLE DEVICE FOR MEASURING THE STATE OF HEALTH OF A USER

(71) Applicant: RUBIX S&I, Toulouse (FR)

(72) Inventor: Jean-Christophe MISFUD, Goudourville (FR)

(73) Assignee: RUBIX S&I, Toulouse (FR)

(21) Appl. No.: 17/435,008

(22) PCT Filed: Mar. 27, 2020
(86) PCT No.: PCT/EP2020/058861

§ 371 Date: Aug. 30, 2021

(30) Foreign Application Priority Data
Mar. 29, 2019 (FR) 1903314

(51) Int. Cl. A61B 5/145; A61B 5/1477; A61B 5/00; H02J 7/00; H01M 10/46; H01M 50/247

(52) U.S. Cl. CPC A61B 5/14517; A61B 5/1477; A61B 5/681; A61B 5/6804; H01M 2220/30; H01M 10/46; H01M 50/247; A61B 2560/0214; H02J 7/0042

(57) ABSTRACT

A portable device for measuring the state of health of a user configured to be carried/worn by the user and comprising at least one measurement unit and at least one electric battery supplying the measurement unit with electricity, said electric battery comprising at least two charging electrodes that are configured to cooperate with a charging device in order to electrically recharge the electric battery, among which at least two of the charging electrodes, referred to as charging and measurement electrodes, are configured so as to be in contact with the sweat of the user and are connected to the measurement unit in order to measure, in cooperation with said measurement unit, at least one physical parameter of the sweat of the user, in order to measure their state of health.

### TECHNICAL FIELD

The present invention is concerned with devices for measuring a user's state of health and that are wearable.

In a known manner, a wearable health measurement device is configured to be worn by a user and allows the measurement of various health indicators of the user to monitor the user's state of health. For this, this device comprises at least one measurement member as well as an electric battery for supplying the measurement member.

A connected watch comprising such a device and configured to be worn on the user's wrist is known from prior art. The measurement member may be an optical sensor for measuring the pulse or the blood oxygen level, or a temperature sensor for measuring the body temperature. The connected watch comprises an electric battery that can be charged by an electric charger. In practice, the battery comprises a USB plug type connector.

In prior art, from patent application FR2912049A1 a connected watch comprising such a device whose measurement members are two test electrodes configured to be in contact with the sweat on the user's wrist is also known. This device further comprises a generator sending an electric current between the test electrodes and a voltmeter measuring the impedance of the user's perspiration. This impedance measurement is used to determine whether the user is stressed or unwell. The electric battery is similar to the one described above.

Additionally, from patent application US2016038055A1, there is known a case mounted to a wristband comprising two electrical contacts in contact with the user's skin, a resistance sensor, a capacitance sensor, a capacitor and an electric battery in order to measure the resistance, capacitance and Galvani potential of the user's skin. Both electrical contacts have to comprise the same material in order to measure the Galvani potential as defined by the International Union of Pure and Applied Chemistry (IUPAC). The drawback of such a case is that it has a large overall size and cost and limited measurement possibilities.

In a similar way, a case comprising two electrodes for measuring a Galvani resistance and a third electrode mounted to the outer face so as to be reachable by the user's finger is known from patent application US20161928561A1. An ECG sensor is used to measure an electrocardiogram between the third electrode and one of the two electrodes on the inner face. Such a case has the same drawbacks as before.

Furthermore, from patent application US2017023518A1, a case comprising several reference electrodes each coupled to an ion-selective field effect transistor for measuring the electrolytic concentration of this ion in the user's sweat is known. Such a case has a large overall size because it requires field effect transistors to make the reference electrodes selective, which are not selective by nature. Further, each electrode allows for a single independent measurement.

A wearable state of health measurement device can thus inform his/her user of his/her state of health immediately. It also meets some of the expectations of users in terms of telemedicine faced with a lack of medical resources. However, such a device has a great overall size and a high cost, which hinders its adoption by the majority of people. The invention thus aims at solving at least some of these drawbacks.

### SUMMARY

The invention relates to a wearable device for measuring a user's state of health configured to be worn by the user, said device comprising at least one measurement member and at least one electric battery electrically supplying the measurement member, said electric battery comprising at least two charge electrodes configured to cooperate with a charge device in order to electrically charge the electric battery.

The invention is remarkable in that at least two of the charge electrodes, so-called charge and measurement electrodes, are configured to contact the user's sweat when the device is worn and are connected to the measurement member in order to measure, in cooperation with said measurement member, at least one physical parameter of the user's sweat, in order to measure his/her state of health.

Advantageously, the charge and measurement electrodes fulfil, on the one hand, a charge function and, on the other hand, a measurement function. Such a dual function advantageously makes it possible to reduce the number of elements as compared to prior art, which limits cost and overall size. Further, advantageously, upon electrical charging via the charge electrodes, the electrodes are in physical contact with the charge device, thus cleaning the surface of the electrodes, which can thus perform relevant measurements.

Preferably, all of the charge electrodes are charge and measurement electrodes. In other words, there are no electrodes dedicated solely to charging. The number of measurements is advantageously optimized for reduced mass and overall size.

Preferably, the charge and measurement electrodes are configured to measure the electrolytic potentials difference in cooperation with the measurement member. Advantageously, the electrolytic potentials difference is a function of the medium in which the charge and measurement electrodes are positioned, that is the user's sweat, as well as the nature of the charge and measurement electrodes.

Preferably, the device comprises at least one calculation member configured to determine at least one health indicator of the user from the electrolytic potentials difference. The determination of the health indicator is performed by algebraic calculation and/or by searching a database.

Preferably, the device comprises at least one comparison member configured to compare the health indicator to at least one reference threshold. The reference threshold is known to the comparison member via theory, via correlations, or via at least one previously determined health indicator. The comparison of the health indicator to the reference threshold is performed by algebraic calculation and/or by searching a database.

Preferentially, the measurement device and the calculation member belong to the same electronic unit, preferably an electronic board. This reduces the mass and overall size.

Preferentially, the measurement member and the comparison member belong to the same electronic unit, preferably an electronic board. This reduces the mass and overall size.

Preferably, the device comprising at least three charge and measurement electrodes defining at least one reference electrode and at least two test electrodes, the measurement member is configured to measure the electrolytic potentials difference between the reference electrode and each test electrode. The use of multiple test electrodes allows for multiple measurements to be obtained in order to determine several user's health indicators, or to obtain a more relevant and reliable user's health indicator.

Preferably, the reference electrode is unique. Advantageously, the number of measurements is optimized for reduced mass and overall size.

Preferably, at least two test electrodes are of different materials so as to measure at least two different electrolytic potential differences. Advantageously, the electrolytic potentials difference is a function of the nature of the material of the test electrode used for the measurement.

Preferentially, all the test electrodes are made of different materials.

Preferably, the material of at least one charge and measurement electrode comprises at least one metal from the following list: Ti, Zr, Hf, V, Nb, Cr, Mo, W, Mn, Fe, Ru, Co, Rh, Jr, Ni, Pd, Cu, Ag, Au, Al, Sn, Zn and Pt or an alloy comprising a metal from the preceding list or a metal compound from a metal of the preceding list. Advantageously, the material of a charge and measurement electrode conducts electricity to perform the charge function and is adapted for the desired electrolytic potentials difference measurement.

Preferentially, the device comprises at least one measurement electrode. Advantageously, the measurement electrode complements measurements made by the charging and measurement electrodes, to more reliably and accurately measure the user's state of health.

Preferentially, the material of at least one measurement electrode comprises a polymer, ceramic or ion-conducting glass.

The invention also relates to a wristband configured to be worn on the user's wrist and comprising a case fixedly mounted to said wristband, said case comprising an internal face configured to contact the user's wrist when the wristband is worn, and an external face opposite to the internal face, said wristband comprising the device as described above, mounted in the case, the charge and measurement electrodes being mounted to the internal face, so as to contact the sweat (S) on the user's wrist.

The invention also relates to a garment configured to be worn by the user and comprising a reverse side configured to contact the user's skin when the garment is worn, a location opposite to the reverse side, said garment comprising the device as previously described, being attached to the reverse side so as to contact the user's sweat.

Preferentially, the garment is a T-shirt.

Preferentially, the garment is a belt.

Preferentially, the garment is an undergarment.

Preferentially, the garment is a sock.

Preferentially, the garment is a sole.

The invention further relates to a method for measuring the state of health of a user wearing a device as described above, the method comprising a step of measuring by the measurement member in cooperation with the charge and measurement electrodes at least one physical parameter of the user's sweat in order to measure the user's state of health.

Preferably, the method comprises a calculation step, during which the calculation member determines at least one health indicator of the user from the physical parameter of the sweat.

Preferably, the method comprises a comparison step, during which the comparison member compares the health indicator with a reference threshold.

Preferentially, the method comprises a user prevention step, during which the comparison member emits an audible, visual or other alarm when the health indicator I exceeds the reference threshold I-ref so as to inform the user of his/her state of health.

Preferably, the method comprises a charge step, during which the electric battery 3 is connected to a charge device in order to be charged with electricity.

### BRIEF DESCRIPTION OF THE DRAWINGS

The invention will be better understood upon reading the following description, which is given solely by way of example, and refers to the appended drawings given as non-limiting examples, in which identical references are given to similar objects and in which:

FIG. 1 is a schematic representation of a user wearing a wristband according to the invention,

FIG. 2 is a schematic representation of the wristband according to the invention,

FIG. 3 is a schematic representation of the case of the wristband comprising the wearable measurement device of the user's state of health according to the invention and the charge operation of the electric battery,

FIG. 4 is a schematic representation of the case of the wristband comprising the device for wearable measurement of the user's state of health according to the invention and of the discharge operation of the electric battery,

FIG. 5 is a schematic representation of the case of the wristband comprising the wearable device for measuring the user's state of health according to the invention and of the operation of the charge and measurement electrodes in cooperation with the measurement member,

FIG. 6 is a schematic representation of the case of the wristband comprising the wearable device for measuring the user's state of health according to the invention and of the operation of the calculation member and the comparison member,

FIG. 7 is a schematic representation of a T-shirt comprising the wearable device for measuring the user's state of health according to the invention,

FIG. 8 is a schematic representation of a belt comprising the wearable user state of health measurement device according to the invention,

FIG. 9A,

FIG. 9B and

FIG. 9C are a schematic representation of a user wearing the belt according to the invention on the waist, ankle and hind arm respectively and FIG. 10A,

FIG. 10B and

FIG. 10C are schematic representations of the arrangement of the measurement electrodes of the wearable measurement device according to three embodiments of the invention.

It should be noted that the figures set out the invention in detail for implementing the invention, said figures may of course serve to better define the invention where appropriate.

### DETAILED DESCRIPTION

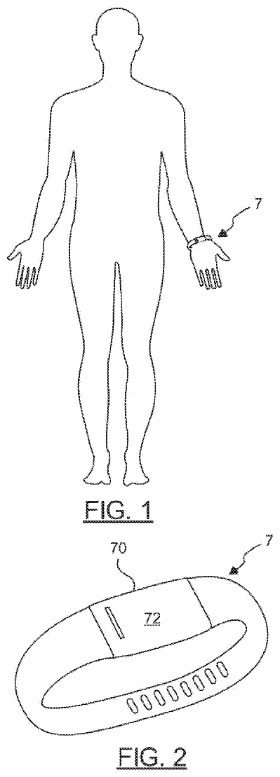

A wearable health measurement device according to the invention will be set forth. With reference to FIGS. 1 to 6, a connected watch comprising a wristband 7 comprising a wearable device according to the invention is set forth. However, this invention is applicable to any garment or the like configured to be worn by the user and in contact with the user's skin, such as a sock, belt, sole, undergarment, adhesive tape, etc.

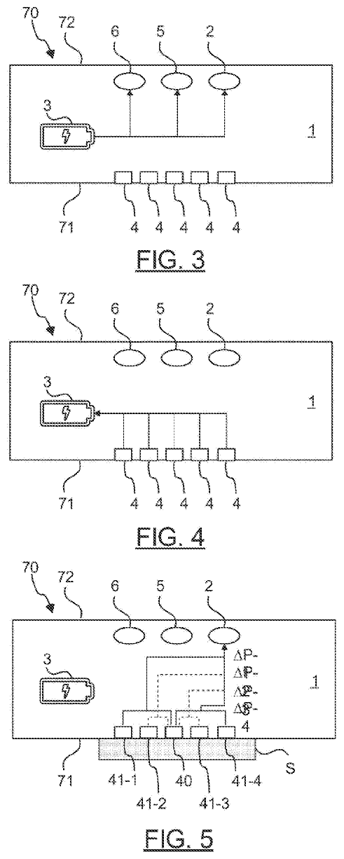

In the example of FIGS. 1 and 2, the wristband 7 is configured to be worn on the user's wrist and comprises a case 70 mounted to the wristband 7. As illustrated in FIG. 3, the case 70 comprises an internal face 71 configured to contact the user's wrist when the wristband 7 is worn, an external face 72 opposite to the internal face and the wearable user state of health measurement device 1 mounted in the case 70. The wristband 7 is known to have a ring shape and comprises a closure system, for example, with notches.

According to the invention, with reference to FIGS. 3 and 4 schematically representing the case 70 in a longitudinal cross-section view, the device 1 comprises a measurement member 2 and an electric battery 3 electrically supplying the measurement member 2. The electric battery 3 comprises in this example five charge electrodes 4 configured to cooperate with a charge device (not represented) in order to electrically charge said electric battery 3. According to the invention, the electric battery 3 comprises at least two charge electrodes 4. The charge device may be an electrical charger connected to the electrical network via a socket as an example. In other words, the electric battery has two modes of operation, namely a charging mode of operation where it is supplied by the charge device (FIG. 4) and a discharging mode of operation where it supplies the measurement device 2 (FIG. 3). It goes without saying that the device 1 may comprise several electric batteries 3, which however increases the overall size and mass of said device 1. Similarly, the device 1 may comprise several measurement devices 2, so as to carry out measurements of different kinds.

According to the invention, with reference to FIG. 5, at least two of the charge electrodes 4 are positioned on the internal face 71 of the case 70 so as to be in contact with the sweat S of the user's wrist. In this example, all five electrodes 4 are positioned on the internal face 71. These charge electrodes 4, hereinafter referred to as charge and measurement electrodes, measure in cooperation with the measurement member 2 at least one physical parameter of the sweat S of the user's wrist, according to a second function. In other words, the charge and measurement electrodes have two functions: a first, charge function in cooperation with the charge device (FIG. 4) and a second, measurement function in cooperation with the measurement device 2 (FIG. 5). In the example shown in FIG. 5, all the charge electrodes 4 are charge and measurement electrodes, in order to advantageously perform a larger number of measurements. It goes without saying, however, that some of the charge electrodes 4 can be dedicated solely to the charge function. Furthermore, in the example shown in FIG. 5, the device 1 comprises five charge electrodes 4, but it goes without saying that the number of charge electrodes 4 is any number. Preferably, this number is large enough to perform various measurements and small enough to limit the overall size and mass.

According to one aspect of the invention, as illustrated in FIG. 5, the measurement member 2 is in the form of a voltmeter connected to the charge and measurement electrodes and is configured to measure a plurality of electrolytic potential differences AP from the user's sweat S. In the example of FIG. 5, the measurement device 2 is unique in order to limit the mass and overall size of the device 1, but it goes without saying that the device 1 may comprise several measurement devices 2. Furthermore, it goes without saying that the measurement device 2 may be in a form other than a voltmeter, such as an ohmmeter or an ammeter by way of example. Accordingly, the measurement device 2 may be configured to measure any physical parameter of the sweat S, other than an electrolytic potentials difference AP, such as the resistance or the electric current associated with this electrolytic potentials difference AP.

In practice, an electrolytic potential corresponds to the potential emitted by the chemical reactivity of one or more chemical species contained in the sweat S and known to the person skilled in the art as ions, with one or more chemical species of a charge and measurement electrode. An electrolytic potentials difference AP thus corresponds to an electric voltage equal to the difference between a first electrolytic potential associated with a first charge and measurement electrode and a second electrolytic potential associated with a second charge and measurement electrode. In the following, a charge and measurement electrode, in particular its measurement function, is described in more detail.

In practice, a charge and measurement electrode comprises a body comprising molecules which react spontaneously, according to a chemical reaction known to the person skilled in the art as a redox reaction, with some of the ions of the sweat S. The pair formed by an ion and a molecule of the body of the charge and measurement electrode reacting together is known to the person skilled in the art as a redox couple. The ions are electrically charged so that the redox reaction produces a spontaneous electrical voltage, corresponding to the electrolytic potentials difference AP.

According to one aspect of the invention and with reference to FIG. 5, the measurement member 2 is thus configured to measure the electrolytic potential difference of a redox couple associated with a first charge and measurement electrode, referred to as the test electrode 41, with respect to that of another redox couple, referred to as the reference redox couple and associated with a second charge and measurement electrode, referred to as the reference electrode 40.

Preferably, as in the example of FIG. 5, one of the charge and measurement electrodes forms a reference electrode 40 while the other charge and measurement electrodes form test electrodes 41, in order to carry out a measurement of the electrolytic potentials difference AP between the reference electrode 40 and each test electrode 41. The overall size and mass of the device 1 are thus advantageously optimized. However, it goes without saying that several charge and measurement electrodes can form reference electrodes 40. The charge and measurement electrodes are metallic.

According to one aspect of the invention, the material of the charge and measurement electrodes comprises at least one metal from the following list: Ti, Zr, Hf, V, Nb, Cr, Mo, W, Mn, Fe, Ru, Co, Rh, Jr, Ni, Pd, Cu, Ag, Au, Al and Sn or an alloy comprising a metal from the preceding list or a metal compound from a metal of the preceding list. For example, the charge and measurement electrode comprises a renewable layer of ruthenium dioxide RuO2, manganese dioxide MnO₂ or zinc phosphate Zn₃(PO₄)₂.

Preferably, the body of each charge and measurement electrode is made of a different material in order to measure differences in electrolytic potentials AP of different redox couples so as to monitor the user's state of health in a comprehensive and diverse manner. For example, with reference to FIG. 5, the reference electrode 40 is made of platinum, a first test electrode 41-1 is of iron, a second test electrode 41-2 is of magnesium, a third test electrode 41-3 is of silver coated with a silver chloride layer and a third test electrode 41-4 is of zinc. These test electrodes 41 are thus configured to chemically react with the ferrous ions, magnesium ions, chloride ions and zinc ions of the sweat S respectively. However, it goes without saying that the material of several charge and measurement electrodes may be identical.

According to one aspect of the invention, the device 1 further comprises one or more measurement electrodes, solely dedicated to the measurement of electrolytic potential differences P. Preferably, the number of measurement electrodes is reduced to limit the overall size and mass of the device 1. According to one aspect, these measurement electrodes comprise a body whose material is a polymer, ceramic or ion-conducting glass. The materials mentioned are indeed not metallic and cannot be used to achieve charging. Advantageously, these measurement electrodes allow the user's state of health to be measured in a complementary manner to the charge and measurement electrodes.

According to one aspect of the invention, the measurement electrodes, which may also be dedicated to charging, have a large contact surface area with the user's skin so that the measurements of electrolytic potential differences AP, that is potentiometric measurements, are accurate and easily interpretable. According to another aspect, for amperometric measurements, that is the current associated with the electrolytic potentials difference AP, the measurement electrodes, which may also be dedicated to charging, have a small contact surface area with the user's skin so that the measured current densities are high and therefore easily interpretable. Preferably, the measurement electrodes, which may also be dedicated to charging, have different contact surface areas, in order to allow both accurate and easily interpretable potentiometric and amperometric measurements, as well as a greater diversity of measurements.

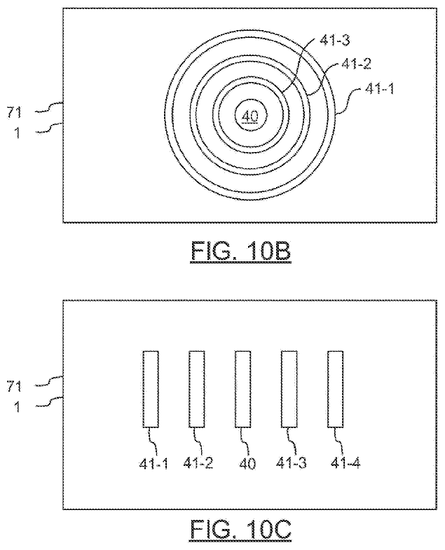

Preferably, with reference to FIGS. 10A and 10B, the contact surfaces of the electrodes with the user's skin are in the form of a disc surrounded by concentric rings—or portions of rings—in order to limit the overall size of the wearable measurement device 1. Alternatively, in the example of FIG. 10C, the contact surfaces of the electrodes with the user's skin are located side by side to limit overall size. Preferably, the distance between two adjacent electrodes is identical for amperometric measurements, especially so that ohmic losses between the reference electrode 40 and the individual test electrodes 41 are identical. In the example shown in FIG. 10A, assuming that the reference electrode 41 has the central disc-shaped contact surface, the ohmic losses between the reference electrode 40 and the test electrodes 41 whose contact surface is in the form of ring portions of the same diameter are identical. Furthermore, the manufacture and assembly of measurement electrodes with concentric contact surfaces is advantageously simple.

Preferably, the device 1 further comprises an enzyme sensor in order to improve accuracy of the measurements of the electrolytic potential differences AP. Of course, the device 1 may comprise other types of sensors helping to improve accuracy of said measurements.

Complementarily, the measurement member 2 may be configured to measure a potentiostatic and/or potentiodynamic polarization resistance—and/or the current and the associated potential difference—by means of at least three measurement electrodes, which may also be dedicated to charging. More precisely, in the potentiostatic mode, the measurement member 2 is configured to maintain a fixed potential between a reference electrode and a test electrode, the third measurement electrode forming an auxiliary electrode. In the potentiodynamic mode, on the other hand, the measurement device 2 is configured to maintain a variable potential with given speed and shape. The role of the three measurement electrodes can advantageously be interchanged to diversify measurements. In the following, a measurement device 2 is considered that is configured solely to measure an electrolytic potentials difference AP, but it goes without saying that this description is valid for a measurement device 2 that is also configured to measure a potentiostatic and/or potentiodynamic polarization resistance.

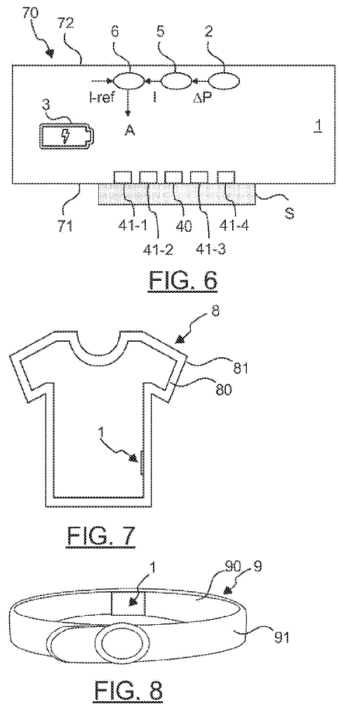

According to one aspect of the invention, as illustrated in FIG. 6, the device 1 also comprises a calculation member 5 configured to determine, from a measurement of electrolytic potential difference AP, a health indicator I of the user. This health indicator I of the user can, for example, be a concentration, a mass or a quantity of a given ion of the sweat S. Taking the example of FIG. 5, the calculation member 5 determines the concentrations of ferrous ions, magnesium ions, chloride ions and zinc ions of the sweat S from the measurements made. Preferably, the calculation member 5 is also configured to determine a health indicator I of the user from a plurality of measurements of electrolytic potential differences AP. Thus, the user's state of health can advantageously be measured in a more diversified manner from a reduced number of measurements and thus of charge and measurement electrodes. For example, the concentration of glutamate ions can be determined in this way, with the help of the enzyme sensor.

Preferably, the measurement device 2 and the calculation member 5 belong to a same entity, such as an electronic board, in order to limit the mass and overall size of the device 1. In the case where the wristband is a connected watch, the measurement device 2 and the calculation member 5 can advantageously form a same entity with the electronic board of said connected watch for the same purpose. In the same way, the electric battery 3 of the device 1 can advantageously form a same entity with the electric battery of said connected watch.

According to another aspect of the invention, the device 1 also comprises a comparison member 6 configured to compare a health indicator I with a reference threshold I-ref. Preferably, said comparison member 6 is configured to warn the user if said reference threshold I-ref is exceeded. For example, if the ferrous ion concentration is below the reference threshold, the comparison member 6 issues an alarm indicating that the user may be suffering from an iron deficiency causing fatigue. For example, if the magnesium ion concentration is higher than the reference threshold, the comparison member 6 issues an alarm indicating that the user may be suffering from diarrhea and vomiting and recommends medical advice. For example, if the glutamate ion concentration is higher than the reference threshold, the comparison member 6 issues an alarm that the user may be suffering from diabetes. Advantageously, the user is warned quickly and can, if necessary, seek treatment as soon as possible. The comparison member 6 may emit an audible, visual, light, computerized or other alarm.

Preferentially, the reference threshold I-ref is predetermined with the aid of prior electrolytic potential difference measurements in order to achieve a reliable, accurate and user-specific comparison. It goes without saying, however, that the reference threshold I-ref can be obtained in any way, by theory or by correlations as an example.

Preferably, the measurement device 2 and the comparison device 6 form a same entity, such as an electronic board, in order to limit mass and overall size of the device 1. In the case where the wristband is a connected watch, the measurement device 2 and the comparison device 6 can advantageously form a same entity with the electronic board of said connected watch for this same purpose.

According to one aspect of the invention, the device 1 further comprises a programming member configured to periodically control measurement of the electrolytic potentials difference AP by the measurement member 2 in cooperation with the charge and measurement electrodes.

According to one aspect of the invention, the device 1 also comprises a recording member configured to store the determined health indicator I in memory.

According to one aspect of the invention, the wristband 7 may be used remotely to measure the user's state of health from a body fluid other than sweat S, such as urine or blood. In other words, the device 1 allows the measurement of a user's state of health from any body fluid of said user. In this configuration, the charge and measurement electrodes have to be placed in contact with said body fluid to perform the measurement. Advantageously, this configuration allows a user to perform an emergency measurement and to be able to benefit from a quick result, without having to move.

An embodiment of the invention has been set forth in which the device 1 is mounted to a wristband configured to be worn on the wrist by the user. Of course, the invention is not limited to this embodiment but encompasses any garment or the like configured to be worn by the user and in contact with the user's skin.

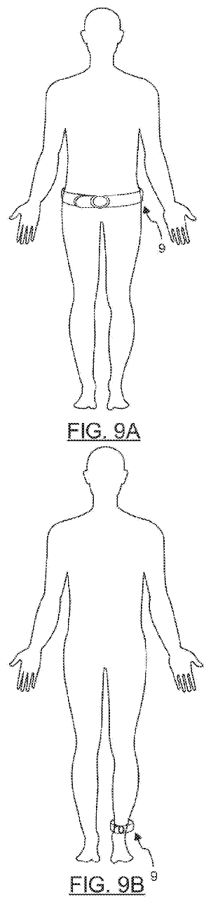

In the example of FIG. 7, the device 1 is thus mounted to a T-shirt 8, configured to be worn by the user and comprising a reverse side 80 configured to be in contact with the user's skin when the T-shirt 8 is worn, and a front side 81 opposite to the reverse side 80. More specifically, the device 1 is configured to be attached to the reverse side 81 of the T-shirt. The example of FIG. 7 is adaptable to any garment in contact with the user's skin, such as an undergarment, sock, trousers, sole, etc.

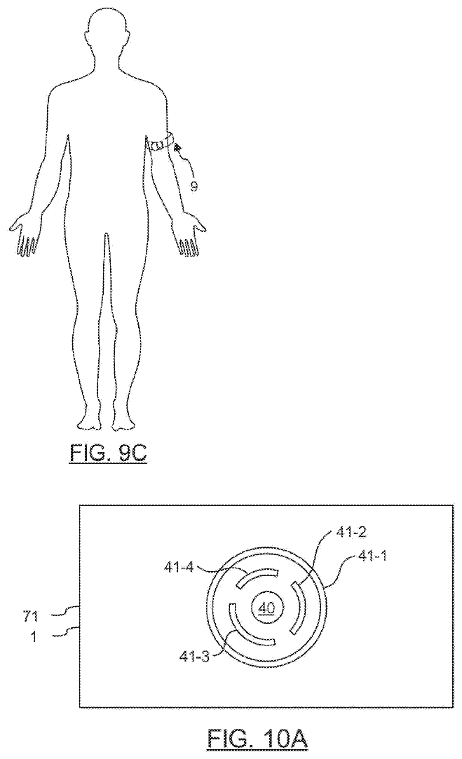

In the example of FIG. 8, the device 1 is mounted to a belt 9, configured to be worn by the user and comprising a reverse side 90 configured to contact the user's skin when the belt 9 is worn, and a front side 91 opposite to the reverse side 90. More specifically, the device 1 is configured to be attached to the reverse side 91 of the belt 9. Such a belt 9 may be worn on the waist, ankle or back of the arm as non-limiting examples represented in FIGS. 9A, 9B and 9C respectively. Similarly, the device 1 may be mounted to an elastic band or adhesive tape, configured to be placed directly on the user's skin.

Advantageously, the device 1 according to the invention may be worn by the user in various ways, depending on the preferences of each user. Further, the device has reduced overall size, mass and cost, which may appeal to the user.

The invention also relates to a method for measuring the state of health of a user wearing a device 1 as described above. This method comprises a measurement step in which the measurement member 2 measures in cooperation with the charge and measurement electrodes at least one physical parameter of the user's sweat S, in order to measure the user's state of health.

According to one aspect of the invention, the measurement member 2 and the charge and measurement electrodes determine at least one electrolytic potentials difference AP.

Preferably, the method comprises a calculation step, in which the calculation member 5 determines at least one health indicator I of the user from the physical parameter of the sweat S.

Preferably, the method comprises a comparison step, during which the comparison member 6 compares the health indicator I with a reference threshold I-ref. Preferably, the method comprises a user prevention step, in which the comparison member 6 issues an alarm when the health indicator I exceeds the reference threshold I-ref so as to inform the user about his/her state of health.

Preferably, the method comprises a charging step, during which the electric battery 3 is connected to a charge device in order to be charged with electricity.

Preferably, the calculation step follows the charging step. Advantageously, during the charging step, the charge and measurement electrodes are in physical contact with the charge device, thereby cleaning the surface of the charge and measurement electrodes, which can thus perform relevant measurements.

The following example considers a user who has a wristband 7 comprising a device 1 according to the invention and who wishes to monitor his/her state of health while jogging. Prior to his jogging, as illustrated in FIG. 3, the user connects the electric battery 3 of the device 1 to an electric charger connected to a wall socket to charge the electric battery 3 via the charge electrodes 4.

Once the electric battery 3 is charged, the user attaches the wristband 7 to his wrist and presses a button to turn on the device 1, as illustrated in FIG. 4. When starting to jog, with reference to FIGS. 5 and 6, the user presses a button to perform a first measurement of electrolytic potential differences AP of his/her sweat S, allowing to determine his/her lactic acid concentration, his/her hydration level and his/her sugar level as examples. This measurement defines his reference threshold I-ref. He/she then programs the device 1 so that a measurement of the differences in electrolytic potentials AP of his/her sweat S is taken every ten minutes during his/her jog. Halfway through the run, the user receives an audible alert from the device 1 indicating that his/her hydration level is low and recommends a break to drink a certain amount of water. At the end of the run, the user takes another measurement and receives an audible alert that his/her sugar level is low and his/her lactic acid concentration is rising, recommending that he/she eats an energy bar and do some stretching to avoid soreness. Once the jog is over, the user presses a button to turn off the device 1 and removes the wristband 7 from the wrist. The user can also keep a history of the measurements for future jogging sessions, allowing the comparison member to refine the reference threshold I-ref to be unique to the user.

1-8. (canceled)

9. A wearable device for measuring a user's state of health, configured to be worn by the user and comprising at least one measurement member and at least one electric battery electrically supplying the measurement member, said electric battery comprising at least three charge electrodes configured to cooperate with a charge device in order to electrically charge the electric battery, wherein at least three of the charge electrodes, called charge and measurement electrodes and defining at least one reference electrode and at least two test electrodes, are configured to be in contact with the user's sweat when the device is worn and are connected to the measurement member in order to measure, in cooperation with said measurement member, at least one physical parameter of the user's sweat, namely the electrolytic potentials difference between the reference electrode and each test electrode, in order to measure the user's state of health.

10. The device, according to claim 9, comprising at least one calculation member configured to determine at least one health indicator of the user from the electrolytic potentials difference.

11. The device according to claim 10, comprising at least one comparison member configured to compare the health indicator to at least one reference threshold.

12. The device, according to claim 9, wherein at least two test electrodes are of different materials so as to measure at least two different electrolytic potential differences.

13. The device, according to claim 9, wherein the material of at least one charge and measurement electrode comprises at least one metal from the following list: Ti, Zr, Hf, V, Nb, Cr, Mo, W, Mn, Fe, Ru, Co, Rh, Ir, Ni, Pd, Cu, Ag, Au, Al, Sn, Zn and Pt or an alloy comprising a metal from the preceding list or a metal compound from a metal of the preceding list.

14. A wristband, configured to be worn on the user's wrist and comprising a case fixedly mounted to said wristband, said case comprising an internal face configured to be in contact with the user's wrist when the wristband is worn and an external face opposite to the internal face, said wristband comprising the device, according to claim 9, mounted in the case, the charge and measurement electrodes being mounted to the internal face, so as to be in contact with the sweat of the user's wrist.

15. A garment, configured to be worn by the user and comprising a reverse side configured to contact the user's skin when the garment is worn, a front side opposite to the reverse side, said garment comprising the device, according to claim 9, attached to the reverse side so as to contact the user's sweat.

16. A method for measuring the state of health of a user wearing the device according to claim 9, the method comprising a step of measuring by the measurement member in cooperation with the charge and measurement electrodes at least one physical parameter of the user's sweat in order to measure the user's state of health.
